(19) United States
(12) Patent Application Publication
(10) Pub. No.: US 2026/0021303 A1
(43) Pub. Date: Jan. 22, 2026

(54) SYSTEMS AND METHODS FOR OBSTRUCTIVE RESPIRATORY EVENT DETECTION AND STIMULATION THERAPY

(71) Applicant: LivaNova USA, Inc., Houston, TX (US)

(72) Inventors: Ohad Israel Balila, Houston, TX (US); Jason Darryl Begnaud, Houston, TX (US); Gerrard Carlson, Houston, TX (US)

(73) Assignee: LivaNova USA, Inc., Houston, TX (US)

(21) Appl. No.: 18/775,590

(22) Filed: Jul. 17, 2024

(51) Int. Cl. A61N 1/36; A61B 5/00; A61B 5/08; A61B 5/113; A61B 5/369; A61N 1/05; A61N 1/372

(52) U.S. Cl. CPC A61N 1/36135; A61B 5/0826; A61B 5/1135; A61B 5/369; A61B 5/4812; A61B 5/4836; A61B 5/686; A61B 5/6867; A61B 5/7264; A61B 5/7282; A61B 5/7285; A61B 5/742; A61N 1/0551; A61N 1/3611; A61N 1/36157; A61N 1/36171; A61N 1/36175; A61N 1/36185; A61N 1/37247; A61B 2560/045; A61B 2562/0219

(57) ABSTRACT

A method includes capturing respiratory data and comparative respiratory data of a patient using a sensor of an implantable medical device, the respiratory data captured during a detection window and the comparative respiratory data captured during a comparative detection window at least partially preceding the detection window. The method further includes comparing the respiratory data to the comparative respiratory data. The method further includes detecting an obstructive respiratory event based on the comparison between the respiratory data and the comparative respiratory data.

### TECHNOLOGY FIELD

The present disclosure relates to devices, systems and associated methods for detecting and treating sleeping disorders. More particularly, the present disclosure relates to devices, systems and methods for detecting and treating obstructive respiratory events during sleep.

### BACKGROUND

Targeted Hypoglossal Nerve (“THN”) stimulation may be used to treat obstructive sleep apnea (“OSA”). Stimulation systems used for THN are generally indicated for the reduction of apneas and/or hypopneas in adult patients with moderate to severe OSA who decline to use or do not tolerate positive airway pressure (“PAP”) therapy for the treatment of OSA.

### SUMMARY

One embodiment relates to a method. The method includes capturing respiratory data and comparative respiratory data of a patient using a sensor of an implantable medical device, the respiratory data captured during a detection window and the comparative respiratory data captured during a comparative detection window at least partially preceding the detection window. The method further includes comparing the respiratory data to the comparative respiratory data. The method further includes detecting an obstructive respiratory event based on the comparison between the respiratory data and the comparative respiratory data.

Another embodiment relates to an implantable medical device. The implantable medical device includes a sensor. The implantable medical device further includes a stimulation lead having a plurality of electrodes. The implantable medical device further includes a pulse generator configured to deliver stimulation to one or more electrodes of the plurality of electrodes of the stimulation lead. The pulse generator includes a processing circuit including a processor and a memory. The memory has instructions stored thereon that, when executed by the processor, cause the processor to capture respiratory data and comparative respiratory data of a patient using the sensor, the respiratory data captured during a detection window and the comparative respiratory data captured during a comparative detection window at least partially preceding the detection window. The instructions further cause the processor to compare the respiratory data to the comparative respiratory data. The instructions further cause the processor to detect obstructive respiratory events during each of a baseline period and one or more stimulation periods based on the comparison of the respiratory data to the comparative respiratory data. The instructions further cause the processor to identify one or more stimulation parameters corresponding to a reduction in the obstructive respiratory events. The instructions further cause the processor to deliver stimulation to the patient using the one or more stimulation parameters.

Another embodiment relates to one or more non-transitory computer-readable media comprising instructions executable by one or more processors to capture respiratory data and comparative respiratory data of a patient using a sensor of an implantable medical device, the respiratory data captured during a detection window and the comparative respiratory data captured during a comparative detection window at least partially preceding the detection window. The instructions are further executable by the one or more processors to compare the respiratory data to the comparative respiratory data. The instructions are further executable by the one or more processors to detect obstructive respiratory events during each of a baseline period, in which no stimulation or a first stimulation intensity is delivered to the patient, and one or more stimulation periods, in which one or more second stimulation intensities are delivered to the patient, based on the comparison of the respiratory data to the comparative respiratory data. The instructions are further executable by the one or more processors to identify one or more stimulation parameters corresponding to a reduction in the obstructive respiratory events based on the detected obstructive respiratory events during each of the baseline period and the one or more stimulation periods.

### BRIEF DESCRIPTION OF THE DRAWINGS

Further features, characteristics, and advantages of the present disclosure will become apparent to a person of ordinary skill in the art from the following detailed description of embodiments of the present disclosure, made with reference to the drawings annexed, in which like reference characters refer to like elements.

FIG. 1 is a schematic view of a system for treating obstructive sleep apnea (“OSA”) including an implantable medical device implanted within a patient, according to an example embodiment.

FIG. 2 is a block diagram showing components of the system of FIG. 1, according to an example embodiment.

FIG. 3 is flowchart of a method for detecting respiratory events, according to an example embodiment.

FIG. 4 is a flowchart of a method for titrating stimulation parameters for treating OSA, according to an example embodiment.

FIG. 5 is a chart of a respiratory waveform and a respiratory event detection signal, according to an example embodiment.

### DETAILED DESCRIPTION

The systems and methods described herein allow for the accurate detection and treatment of obstructive respiratory events. Specifically, the systems and methods described herein utilize accelerometer data captured via an implantable medical device to automatically identify obstructive respiratory events. By tracking these detected obstructive respiratory events over a baseline period and various titration periods, the systems and methods allow for the identification and programming of therapy parameters that produce a reduction in obstruction respiratory events, and thus effective treatment of obstructive sleep apnea.

In some instances, hypoglossal stimulation devices are manually programmed. This manual programming may require the presence and expertise of a sleep professional trained both on the particular device used and also in sleep monitoring. One approach involves a trained user (e.g., a sleep professional) recognizing sleep stages, reductions in flow indicative of obstructive events like apneas and hypopneas, drops in oxygen saturation, arousals, wakefulness, and improvements in respiration characteristics to arrive at a set of contacts and stimulation parameters that should reduce the rate of apneas and/or hypopneas; thus also leading to a reduction in arousals. Accordingly, reducing the complexity of the titration system through any level of automation will reduce the cognitive burden of the user. Further, optimizing the therapy for a patient may require multiple visits and/or sleep studies under a manual approach. Reducing the number of visits and the complexity of titration per visit makes the system easier to use for all involved.

The systems and methods described herein address these issues by allowing for the automatic detection, tracking, and monitoring of obstructive respiratory events, as well as the automatic titration of various therapy parameters to identify effective combinations of therapy parameters (e.g., stimulation parameters, activated stimulation electrodes) for reducing obstructive respiratory events. Specifically, the systems and methods are able to achieve this automation through use of a unique comparison between near-term accelerometer data and long-term accelerometer data that is used to identify if the patient's chest acceleration has reduced, which may be indicative of an obstructive respiratory event. Beneficially, the methods described herein do not utilize a total amplitude of chest movement to detect obstructive respiratory events, which can fluctuate when the patient changes body positions. Instead, the comparison between the near-term accelerometer data and the long-term accelerometer data utilized is capable of accurately detecting obstructive respiratory events regardless of body position.

Additionally, because the obstructive respiratory events are detected using an accelerometer, in some embodiments, the patient's body position can beneficially be monitored and the obstructive respiratory events can be tracked against the patient's body position to determine which positions result in the highest number of obstructive respiratory events and which therapy parameters result in the highest reduction of obstructive respiratory events in each body position. In some instances, the patient's sleep stages can additionally be monitored via an electroencephalogram (“EEG”) sensor and tracked against the obstructive respiratory events to determine which sleep stages have the highest number of obstructive respiratory events and which therapy parameters result in the highest reduction of obstructive respiratory events in each sleep stage.

Accordingly, the systems and methods described herein further allow for the identification and application of scenario- or context-specific stimulation, in some embodiments. For example, some embodiments of the systems and methods described herein may allow for optimal therapy parameters (e.g., therapy parameters associated with a largest reduction of obstructive respiratory events) to be identified for different body positions (e.g., supine, prone, the patient lying on their side) and/or different sleep stages (e.g., NREM, REM) to allow for different therapy parameters to be applied to the patient in different body positions and/or sleep stages, thereby providing improved efficacy of obstructive sleep apnea treatment.

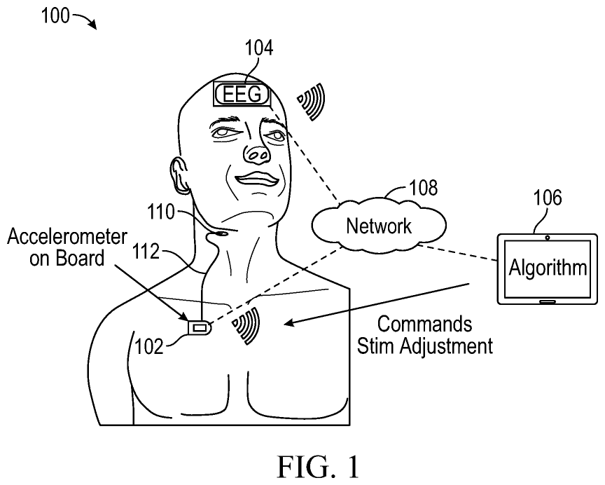

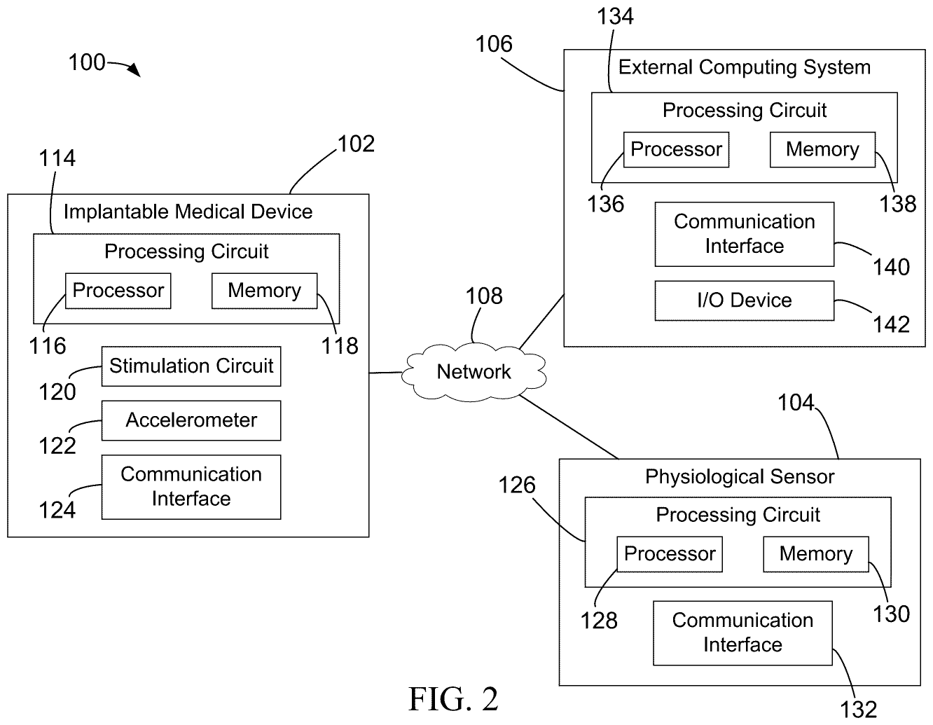

Referring now to FIGS. 1 and 2, a system 100 for treating obstructive sleep apnea (“OSA”) is shown, according to an example embodiment. The system 100 includes an implantable medical device (“IMD”) 102 and a physiological sensor 104, each in communication with an external computing system 106 via a network 108. In some instances, the network 108 can be a near-field communication network, a Bluetooth network, a Wi-Fi network, a radio-frequency-based network a local area network, or any other suitable type of network for transmitting data between different components of the system 100.

The IMD 102 is configured to deliver stimulation to a patient via one or more stimulation electrodes 110 of a stimulation lead 112. In some instances, the IMD 102 is a hypoglossal nerve stimulator configured to be implanted within the upper chest area (right or left side) of the patient, and the one or more stimulation electrodes 110 are attached to and configured to deliver stimulation to the hypoglossal nerve.

In some instances, the one or more stimulation electrodes 110 include a plurality of stimulation electrodes 110 attached to the hypoglossal nerve. For example, the stimulation lead 112 may include six independent electrode contacts within a cuff and spaced circumneurally around the hypoglossal nerve before the nerve branches off to the various muscle groups of the tongue to allow for selective stimulation of desired muscle groups. As will be described further below, placement of the IMD 102 within the upper chest allows for accurate detection of both (1) acceleration caused by the patient's inspiration and exhalation and (2) patient body position.

As shown in FIG. 2, the IMD 102 includes a processing circuit 114 having a processor 116 and a memory 118. The memory 118 has instructions stored thereon that, when executed by the processor 116, cause the processor 116 to perform various functions described herein. The IMD 102 further includes a stimulation circuit 120 configured to selectively deliver stimulation to the patient. For example, the stimulation circuit 120 includes a pulse generator configured to produce and apply stimulation to a hypoglossal nerve of the patient via the one or more stimulation electrodes 110 of the stimulation lead 112.

The IMD 102 further includes an accelerometer 122 (shown in FIG. 2). For example, the accelerometer 122 may be contained and controlled by embedded firmware within the IMD 102. In some instances, the accelerometer 122 is a 3-axis accelerometer configured to record the patient's chest acceleration during use. In some instances, the patient's chest acceleration is dependent on a respiration rate of the patient (e.g., breaths per minute) and a tidal volume of the patient (e.g., how deeply the patient is breathing). For example, if either the respiration rate or the tidal volume of the patient is reduced while the other remains constant (or both respiration rate and tidal volume are reduced), the patient's overall chest acceleration will decrease. Alternatively, if either the respiration rate or the tidal volume of the patient is increased while the other remains constant (or both respiration rate and tidal volume are increased), the patient's overall chest acceleration will increase.

In some instances, the raw acceleration data is utilized by an algorithm or other control logic running onboard the IMD 102 (e.g., via the processing circuit 114). In some other instances, the raw acceleration data is streamed to the external computing system 106 to be processed. As will be described herein, the sampled acceleration data may be used to determine whether the patient's chest acceleration is indicative of normal respiration or an obstructed respiratory event (e.g., obstructive apnea, obstructive hypopnea, or a respiratory effort-related sleep arousal (“RERA”)).

The IMD 102 further includes a communication interface 124 configured to communicate with the physiological sensor 104 and the external computing system 106 (e.g., over the network 108). For example, the communication interface 124 may be configured to transmit acceleration data (e.g., associated with the patient's chest acceleration and indicative of the patient's breathing) to the external computing system 106. The communication interface 124 may further be configured to receive various device control instructions (e.g., stimulation parameters, titration parameters) from the external computing system 106 or other programming devices.

The system 100 further includes a physiological sensor 104 configured to monitor one or more physiological signals of the patient. In some instances, as shown in FIG. 1, the physiological sensor 104 may be an EEG sensor configured to monitor the patient's brain activity (e.g., to determine what sleep stage the patient is in). In some other instances, the physiological sensor 104 may be a respiratory inductance plethysmography (“RIP”) belt configured to monitor the patient's respiration (e.g., in addition or alternative to the accelerometer-based methods described herein). In some other instances the physiological sensor 104 may be various other types of physiological sensors configured to monitor various other physiological signals of the patient. Further, in some instances, the system 100 may include a plurality of physiological sensors configured to monitor a plurality of different physiological signals of the patient, as desired for a given application.

The physiological sensor 104 similarly includes a processing circuit 126 having a processor 128 and a memory 130 having instructions stored thereon that, when executed by the processor 128, cause the processor 128 to perform various functions described herein. The physiological sensor 104 further includes a communication interface 132 configured to communicate with the IMD 102 and the external computing system 106 (e.g., over the network 108). For example, the communication interface 132 may be configured to transmit physiological data to the implantable medical device 102 and/or the external computing system 106 via the network.

In some instances, the external computing system 106 comprises a tablet, a desktop computer, a laptop computer, a smart phone, or any other suitable device capable of receiving and transmitting data and user inputs. In some instances, the external computing system 106 may be an at-home patient computing device configured to be located near the patient (e.g., at the patient's bedside). In other instances, the external computing system 106 may be a healthcare provider's computing device configured to be utilized by a healthcare provider in a location that is remote from the patient (e.g., in the healthcare provider's office).

The external computing system 106 similarly includes a processing circuit 134 having a processor 136 and a memory 138 having instructions stored thereon that, when executed by the processor 136, cause the processor 136 to perform various functions described herein. For example, as referenced above, in some instances, processing of the captured accelerometer and/or other physiological data may be performed locally on the IMD 102 (e.g., via the processing circuit 114) or externally on the external computing system 106 (e.g., via the processing circuit 134). Accordingly, the external computing system 106 further includes a communication interface 140 configured to communicate with the IMD 102 and the physiological sensor 104 (e.g., over the network 108). The external computing system 106 further includes one or more input/output (“I/O”) devices 142 configured to allow for input from users and for the output of information to users. For example, the I/O devices 142 may comprise a mouse and keyboard, a display, a touchscreen, or any other suitable I/O devices, as desired for a given application.

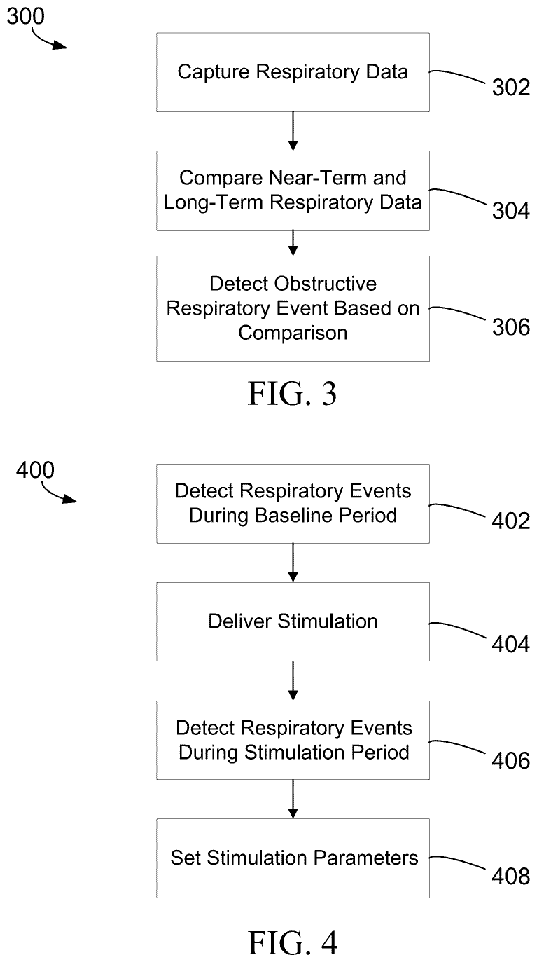

Referring now to FIG. 3, a method 300 for detecting respiratory events is shown, according to an example embodiment. In some instances, the method 300 is performed locally by the IMD 102 (e.g., the processing circuit 114). In some other instances, the method 300 is perform partially by the IMD 102 and partially by the external computing system 106.

The method 300 begins with capturing respiratory data, at step 302. For example, the IMD 102 captures acceleration data associated with the patient's chest acceleration and indicative of the patient's respiration using the accelerometer 122. In some instances, the IMD 102 (e.g., the processing circuit 114) receives the raw acceleration data and smoothes the data (e.g., using one or more data filtering or other smoothing techniques configured to reduce signal noise).

Once the respiratory data has been captured, at step 302, near-term respiratory data is compared to long-term respiratory data, at step 304. For example, in some instances, the acceleration data is continuously captured (e.g., via the accelerometer 122) from the patient and assessed by comparing near-term data (e.g., present data, foreground data) indicative of a current or near-current respiration state with long-term data (e.g., baseline data, background data) indicative of a long-term averaged respiration state. By comparing the near-term data with the long-term data, the IMD 102 can determine whether the near-term data is indicative of normal breathing or of an obstructive respiratory event. In some instances, the acceleration data being captured and assessed continuously may mean constantly for a given period of time, at continuous pre-determined intervals of time (e.g., every second, every thirty seconds, every minute), or according to any other continuous or repetitive timeframe-based monitoring scheme desired for a given application.

In some instances, the near-term respiratory data is respiratory data captured within a moving near-term detection window or timeframe encompassing an immediately preceding near-term period of time. For example, in some instances, the moving near-term detection window or timeframe continuously captures instantaneous or near-instantaneous near-term respiratory data (e.g., chest acceleration data from less than half of a preceding respiration cycle). In some instances, the long-term data is comparative respiratory data captured within a moving long-term detection window or timeframe encompassing a longer period of time preceding the near-term period of time (e.g., a thirty second period preceding the moving near-term detection window or timeframe). For example, in some instances, the moving long-term detection window or timeframe captures multiple respiration cycles. By capturing multiple respiration cycles, the moving long-term detection window or timeframe is unaffected (or less affected) by troughs and peaks in the respiratory data (e.g., caused by the patient's inhalations and exhalations).

In some instances, the long-term detection window is separated from the near-term detection window by a separation amount of time configured to allow for accurate differentiation between the long-term data and the near-term data. For example, in some instances, the moving near-term detection window may encompass respiratory data captured in the last five seconds and the moving long-term detection window may encompass respiratory data captured during a thirty second period ending five seconds prior to the near-term detection window (e.g., a period extending from forty seconds ago to ten seconds ago). In some instances, the long-term detection window may partially overlap with and partially precede the near-term detection window.

To compare the near-term data and the long-term data, a standard deviation or variance (or any other measure of signal change or variation) of the accelerometer samples in each of the near-term detection window and the long-term detection window is calculated. It will be appreciated that utilizing the standard deviations or variances allows for accurate comparisons to be made despite alternating inhalation and exhalation acceleration directions. The standard deviations or variances are then used to calculate a ratio of the near-term data standard deviation or variance over the long-term data standard deviation or variance. This ratio is then used to determine various aspects related to the patient's respiration.

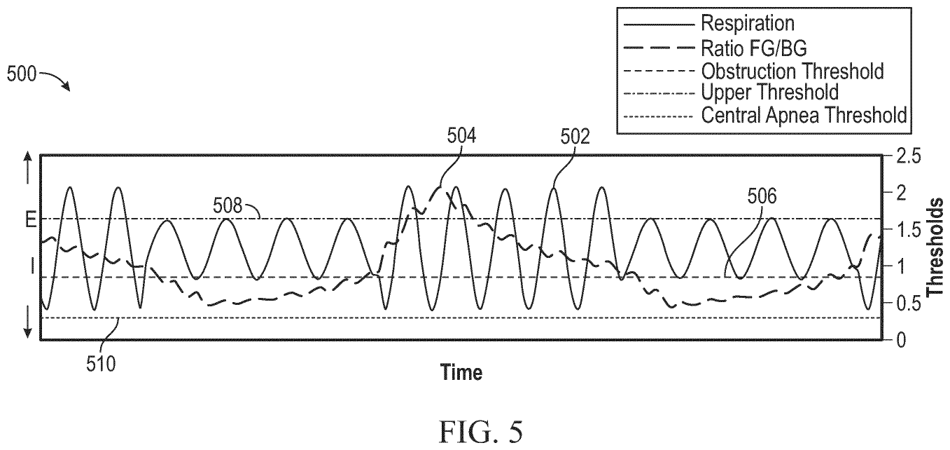

As an example, FIG. 5 shows a chart 500 including a respiratory waveform 502 and a corresponding standard deviation or variance ratio signal 504 of the near-term detection window over the long-term detection window. When the standard deviation or variance ratio is approximately 1, the ratio implies that the captured respiratory data in the near-term detection window is representative of acceptable or otherwise normal respiration. That is, the near-term acceleration data has a standard deviation or variance that is approximately equal to the long-term acceleration data, so no noticeable change has occurred that would indicate a respiratory event.

If the ratio is less than one, the patient's overall chest acceleration was lower during the near-term detection window as compared to the long-term detection window, which may be indicative of an obstructive respiratory event (e.g., because the near-term standard deviation or variance, and thus overall chest acceleration, has reduced compared to the historical reference). Accordingly, in some instances, a lower obstructive threshold 506 (e.g., 0.6, 0.7, 0.8) is set that corresponds to an obstructive respiratory event. As such, if the ratio drops below the lower obstructive threshold 506, the system 100 (e.g., the IMD 102 or the external computing system 106) detects an onset of a potential obstruction or obstructive respiratory event.

If the ratio is greater than 1, the patient's overall chest acceleration was higher during the near-term detection window as compared to the long-term detection window, which may be indicative of an arousal event (e.g., the patient waking up, a strong return to breathing (compensation) due to the arousal mechanism and the patient's body's need for replenishing blood oxygen levels). Accordingly, in some instances, an upper arousal threshold 508 (e.g., 1.7) is set that corresponds to an arousal event. As such, if the ratio rises above the upper arousal threshold 508, the system 100 (e.g., the IMD 102 or the external computing system 106) detects an arousal event. In some instances, if an arousal event occurs after a potential obstruction or obstructive respiratory event, the arousal event may be used as confirmation that the potential obstruction or obstructive respiratory event is, in fact, an obstruction or obstructive respiratory event.

Additionally, if the ratio is significantly less than one (e.g., approaching zero), the patient's chest has stopped moving or nearly stopped moving, which may be associated with a central apnea, as opposed to an obstructive respiratory event. That is, during an obstructive respiratory event, the chest generally continues to move (although at a reduced amount compared to normal), but during a central apnea event, the chest generally does not move at all. Accordingly, in some instances, a lower central apnea threshold 510 (e.g., 0.1, 0.2, 0.3) can be set, lower than the lower obstructive threshold 506, that corresponds to a central apnea event. As such, if the ratio drops below the lower central apnea threshold 510, the system 100 (e.g., the IMD 102 or the external computing system 106) determines that the respiratory event is a central apnea event and not an obstructive respiratory event.

Accordingly, once the near-term respiratory data is compared to long-term respiratory data, at step 304, one or more obstructive respiratory events are detected, at step 306. For example, if the ratio drops below the pre-defined lower threshold, a potential obstructive respiratory event onset is flagged or otherwise annotated. Then, if the ratio subsequently crosses the upper threshold, the event is logged as a detected obstructive respiratory event. For example, the strong return to breathing paired with the initial lower amount of chest acceleration may confirm that an obstructive respiratory event occurred.

In some instances, to qualify the respiratory event as an obstructive respiratory event, the respiratory event must meet certain duration criteria. For example, in some instances, if the respiratory event lasts longer than a predetermined duration threshold (e.g., 10 seconds), the respiratory event qualifies and is logged as an obstructive respiratory event. On the other hand, if the respiratory event does not last as long as the predetermined duration threshold, the respiratory event does not qualify and is not logged as an obstructive respiratory event. In some instances, the duration of each respiratory event (e.g., obstructive respiratory events, non-obstructive respiratory events, arousal events, central apnea events) is tracked and accessible by a user (e.g., the patient, a healthcare provider) of the system 100 (e.g., via the external computing system 106).

It should be appreciated that the ratio thresholds (e.g., the lower obstructive threshold 506, the upper arousal threshold 508, and the central apnea threshold 510) and the duration threshold described above are provided as examples. In some instances, a user of the system 100 may set these thresholds differently and/or may set additional or fewer thresholds, as desired for a given application.

Accordingly, the method 300 can be used to effectively monitor, detect, and track obstructive respiratory events. By using the ratio between near-term accelerometer data and long-term accelerometer data from moving detection windows, the method 300 automatically compensates for changes in body position that affect overall chest movement and acceleration and which could reduce detection accuracy using other devices (e.g., using a respiratory inductance plethysmography (“RIP”) belt) or methods (e.g., using a calculated total chest movement amplitude).

Additionally, unlike RIP belts and other respiratory sensors, an accelerometer is sensitive to gravity and therefore positional change of the patient's body. Body position is generally a factor in the presence and/or severity of obstructive respiratory events. For example, due to the force of gravity, the supine position can bring about more obstructions and is typically a more difficult position to treat with hypoglossal nerve stimulation. Accordingly, in addition to detecting obstructive respiratory events, the accelerometer 122 of the IMD 102 can also be used to track the patient's body position. For example, the accelerometer can first be calibrated to the patient's chest as it resides under the skin after implantation. Then, thresholds can be established that indicate crossing from one body position to another. In some instances, if the IMD 102 moves after implantation, the IMD 102 can be recalibrated using a manual calibration or autonomous algorithmically driven calibration. In any case, the method 300 can further incorporate this body position detection to increase accuracy of the event detection and to track detected events by body position to provide additional context about the patient's obstructive respiratory events.

For example, if a position change is detected during accelerometer data collection, the method 300 may further include repopulating the long-term detection window before then repopulating the near-term detection window. This repopulation of data allows for standard deviations or variances of the long-term data in one position (e.g., supine) to be separated from long-term data in a different position (e.g., prone) to account for differences caused by the different body positions on the accelerometer data, and thereby filter out errant ratio calculations and ensure accurate detection of obstructive respiratory events in each body position.

Further, in some instances, detected obstructive respiratory events are tracked and logged by body position. For example, certain body positions (e.g., a supine position), may be associated with higher amounts of obstructive respiratory events than others for a given patient. Accordingly, the method 300 allows for tracking how many obstructive respiratory events a given patient has in various body positions.

In some instances, the obstructive respiratory events are logged and inform an index (e.g., the respiratory disturbance index (“RDI”) or a similar index) which accounts for obstructive apneas, hypopneas, RERAs, and/or other obstructive respiratory events. Because these events are generally caused by obstruction of the airway, these events can be used to inform an assisted titration scheme or an automated titration scheme, as discussed below.

Various additional events may be tracked and logged as well as obstructive respiratory events. For example, in some instances, regardless of the ratio crossing the lower threshold, if the ratio crosses the upper threshold, the event is logged as a potential arousal event. Tracking these arousal events over time can better inform the arousal index for the night. Further, in addition to body position, detected respiratory events can also be tracked by time of night, by sleep stage (e.g., as measured by the physiological sensor 104), or by any other suitable metric to provide additional context around the detected events. In some instances, tracking and logging the events by body position and/or sleep stage may be helpful for determining how to effectively treat the patient (e.g., via stimulation as discussed below) in different body positions and/or sleep stages.

Central apnea events may also be tracked and/or filtered out, as desired for a given application. For example, central apnea events may necessitate different types of treatments as compared to obstructive apnea events. As such, when discriminated from the obstructive events (e.g., obstructive apneas, hypopneas, and RERAs), these central apnea events can be summarized separately for logging and reporting purposes. Additionally, because the hypoglossal nerve stimulation of the IMD 102 is generally aimed at preventing obstructions of the patient's airway (e.g., by stimulating the muscles of the tongue to move the tongue), in some instances, the central apnea events may be completely excluded from stimulation and titration decisions made by the system 100 (e.g., the IMD 102 or the external computing system 106).

In some instances, the ratio thresholds and/or the duration threshold associated with the obstructive respiratory events (as well as the onset of respiratory events, the arousal events, and the central apnea events) described herein can be adjusted to account for different contextual information or scenarios. That is, in some instances, different ratio thresholds and/or duration thresholds may provide more accurate detection in different body positions, at different times of night, in different stages of sleep, etc., and the system 100 (e.g., the IMD 102 and/or the external computing system 106) may automatically adjust the ratio and/or duration thresholds based on body position and/or stages of sleep.

It should be appreciated that, while the method 300 described above utilizes acceleration data as the respiratory data, the method 300 can similarly be implemented using other types of sensor data capable of revealing or otherwise detailing respiratory interval information (e.g., respiration sensor data, airflow sensor data, chest expansion measurement data).

Referring now to FIG. 4, a method 400 for titrating stimulation parameters for treating obstructive sleep apnea (“OSA”), according to an example embodiment. In some instances, the method 400 can be performed automatically by the IMD 102 and/or the external computing system 106. In some other instances, the method 400 can be performed partially automatically by the IMD 102 and/or the external computing system 106 and partially in response to input provided by a user (e.g., the patient and/or a healthcare professional) via the external computing system 106.

The method 400 begins with detecting respiratory events during a baseline period, at step 402. For example, in some instances, the method 300 discussed above, with respect to FIG. 3, is used to detect the respiratory events during the baseline period. In some instances, the baseline period is a period in time after implantation of the IMD 102 in which the patient is not receiving any stimulation or other treatment and various baseline information about the patient is collected. In some other instances, the baseline period is a period in time after implantation of the IMD 102 where a first stimulation intensity (e.g., a first set of stimulation parameters) is delivered to the patient to be later compared to a second stimulation intensity (e.g., a second set of stimulation parameters). In either case, during the baseline period, the system 100 (e.g., the IMD 102 and/or the external computing system 106) track the respiratory events and log various associated information pertaining to the respiratory events (e.g., body position, sleep stage, time of night). In some instances, the baseline period may be a single night, a week, a month, several months, or any other time period suitable for gathering baseline information about the patient.

Once the baseline respiratory events and associated information have been collected during the baseline period, at step 402, stimulation is delivered to the patient via the IMD 102 and various stimulation parameters are titrated or otherwise modified over or during one or more stimulation periods, at step 404, and respiratory events are detected for the one or more stimulation periods, at step 406. For example, in some instances, the IMD 102 may be configured to automatically begin titrating various stimulation parameters according to one or more pre-programmed titration schemes after the baseline period. In some other instances, the patient may visit a sleep clinic in which a healthcare may program (e.g., using an external programming system or other programming device configured to communicate with the IMD 102) the IMD 102 to titrate the various stimulation parameters. In some instances, the titration period may be a single night. In some instances, the titration period may be significantly longer, such as a week, a month, several months, a year, etc.

During the one or more titration periods, the various stimulation parameters are slowly increased over the course of corresponding titration periods. During this time, the patient's respiratory events are continuously tracked and logged. The stimulation parameters titrated over the titration periods can include current amplitude, frequency, pulse width, duty cycle, etc. In some instances, various additional therapy parameters can be varied during the titration period as well. For example, in some instances, stimulation parameters can be titrated for different stimulation electrodes at different times and/or at different rates to observe how different stimulation electrodes and/or combinations of stimulation electrodes affect the detected respiratory events of the patient.

In some instances, in addition to the respiratory events, the system 100 (e.g., the IMD 102 and/or the external computing system 106) tracks various additional patient information during the titration period. In some instances, the patient wears the physiological sensor 104 while sleeping to allow for the monitoring of one or more physiological signals of the patient during the titration period. For example, as described above, the physiological sensor 104 may be an EEG device that streams live raw EEG data to the IMD 102 and/or the external computing system 106. Accordingly, using this raw data, the IMD 102 and/or the external computing system 106 can determine the patient's sleep stages in near real-time to provide additional context around the patient's detected respiratory events (e.g., which sleep stage the patient is in when a given respiratory event is detected).

In some instances, various additional information may be collected pertaining to the patient during the titration period, such as the patient's heart rate, heart rate variability, oxygen saturation, respiratory flow, respiratory pressure, thoracic and abdominal effort, respiratory inductance plethysmography (RIP) data, body temperature, electromyography (EMG) data, etc., to provide additional context around the detected respiratory events. The patient may further record each night of sleep quality in an electronic diary or using a general wellness sleep device (e.g., a wrist-worn smart watch or a smart ring) and/or application.

Accordingly, by tracking the detected respiratory events and the additional patient information during the titration period, the system 100 (e.g., the IMD 102 and/or the external computing system 106) can determine which stimulation electrodes (e.g., which of the stimulation electrodes 110) at which stimulation levels produced the greatest reduction in detected obstructive respiratory events compared to the baseline collection window. In some instances, the external computing system 106 can generate and display one or more reports including detected respiratory events categorized, for example, by activated electrode contacts used (e.g., a subset of the total number of electrode contacts), by body position of the patient, by sleep stage of the patient, by stimulation parameters used, by time period (e.g., overall, per night, per hour), or any other type of categorization. In some instances, the report further includes an indication of the stimulation electrodes and/or stimulation levels that produced the greatest reduction in detected obstructive respiratory events.

After tracking the detected respiratory events and the additional patient information for the titration period, stimulation therapy parameters are set for the IMD 102, at step 408. For example, in some instances, the IMD 102 and/or the external computing system 106 automatically program the IMD 102 to deliver stimulation to the patient at the identified stimulation parameters using the identified stimulation electrodes that produced the greatest reduction in detected obstructive respiratory events. In some other instances, a healthcare professional can program the therapy parameters to be delivered to the patient (e.g., via the external computing system 106 or another external programming system or device).

In some instances, the therapy parameters may remain constant throughout the night as the patient sleeps. In some other instances, the therapy parameters can be programmed to change responsive to various contextual scenarios throughout the night. For example, based on the comparison of the baseline period to the various titration periods in view of different situational scenarios, the system 100 (e.g., the IMD 102 and/or the external computing system 106) may determine that a first set of therapy parameters works best (e.g., has the greatest reduction in detected obstructive respiratory events) in a first body position (e.g., supine), but that a second set of therapy parameters works best in a second body position (e.g., the patient laying on their side). Similarly, the system 100 (e.g., the IMD 102 and/or the external computing system 106) may determine that a first set of therapy parameters works best in a first sleep stage (e.g., NREM), but that a second set of therapy parameters works best in a second sleep stage (e.g., REM). In some instances, the system 100 (e.g., the IMD 102 and/or the external computing system 106) may determine that it is best to not apply stimulation in certain scenarios (e.g., while the patient is in REM sleep, during a central apnea, or lying on their side). Accordingly, in some instances, the IMD 102 is programmed with a pre-set number of therapy parameter sets for use in different contextual settings (e.g., in different body positions, in different sleep stages).

In some instances, instead of the titration and/or the programming of the various therapy parameters being automated, the titration and/or the programming may be a manual process. For example, in some instances, the patient may travel to a sleep study facility for a sleep study and a user (e.g., a healthcare provider) may modify and/or otherwise adjust various therapy parameters over the course of a night (or several nights) while monitoring various information pertaining to the patient (e.g., displayed via the I/O device 142 of the external computing system 106). For example, in some instances the user may be provided with a real-time or near real-time chart, similar to the chart 500 shown in FIG. 5.

Accordingly, the user may monitor the patient's respiratory waveform, the patient's ratio signal (e.g., the ratio between the near-term standard deviation or variance and the long-term standard deviation or variance described herein), and the various thresholds being utilized for respiratory event detection (e.g., threshold 506, threshold 508, threshold 510). The user may additionally be displayed various other patient information, such as, for example, the patient's sleep stage (assessed using EEG and chin EMG), respiratory flow, oxygen saturation, thoracic and abdominal effort, etc. The user may further be provided with the reports discussed above categorizing detected events by body position and/or sleep stage. The user may also be presented with an indication of which stimulation electrodes and which stimulation parameters resulted in what levels of reduction in obstructive respiratory events.

As described above, the method 400 for titrating stimulation parameters titrates and/or sets stimulation parameters by revealing or otherwise identifying changes in the number of detected obstructive respiratory events (e.g., which may be indicative of sleep apnea events and/or the patient's underlying disease substrate) resulting from differing stimulation parameters and/or intensities to make positive conclusions or assumptions regarding adequate nerve capture and/or titration thresholds achieved by the stimulation therapy.

While the system 100 described herein uses a particular respiratory event detection method (e.g., method 300) and a particular titration/parameter setting method (e.g., method 400), it should be appreciated that various other methods may be used without departing from the scope of the present disclosure. For example, in some instances, instead of an internally integrated accelerometer within the IMD 102 being used, an external accelerometer could be used to collect the raw chest acceleration data described herein.

In some instances, a RIP belt and system may be utilized to detect respiratory events, and the therapy parameters can similarly be subsequently programmed to achieve the highest reduction in detected obstructive respiratory events. In some instances, the IMD 102 may be configured to measure a heart rate variability and/or a transthoracic impedance of the patient (e.g., measured between the one or more electrodes 110 and the can of the IMD 102), which could similarly be used to detect or inform the detection of obstructive respiratory events for use in programming the therapy parameters to be delivered to the patient, as described herein. In some instances, the external computing system 106 may be configured to visually identify obstructive respiratory events (e.g., via a camera and/or a sensor on or near the patient), which can similarly be used to detect obstructive respiratory events for use in programming the therapy parameters to be delivered to the patient, as described herein.

In some instances, in addition to titrating the stimulation parameters and modifying which electrodes are applying stimulation, the system 100 can be used to determine whether modifying stimulation (e.g., turning on the stimulation, increasing a current amplitude delivered to the patient) during a potential obstructive respiratory event results in a reduction of detected obstructive respiratory events. For example, if, during the baseline period, the average duration of a detected obstructive respiratory event is fifteen seconds long, the system (e.g., the IMD 102 and/or the external computing system 106) can cause the IMD 102 to apply or adjust stimulation (e.g., to a higher intensity) a predetermined amount of time after the onset of a potential obstructive respiratory event (e.g., seven seconds). The system can then monitor the patient and, if the number of obstructive respiratory events is reduced or the average duration of the obstructive respiratory events is reduced, the system or a user (e.g., a healthcare professional) can program the IMD 102 to automatically apply or modify stimulation delivered to the patient in response to detected potential obstructive respiratory events.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with processing circuits employing rule-based logic and other logic to accomplish the various connection steps, processing steps, comparison steps and decision steps.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be any of X; Y; Z; X and Y; X and Z; Y and Z; or X, Y, and Z. Thus, such conjunctive language is not generally intended to imply that certain embodiments require at least one of X, at least one of Y, and at least one of Z to each be present.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed:

1. A method comprising:
capturing respiratory data and comparative respiratory data of a patient using a sensor of an implantable medical device, the respiratory data captured during a detection window and the comparative respiratory data captured during a comparative detection window at least partially preceding the detection window;
comparing the respiratory data to the comparative respiratory data; and
detecting an obstructive respiratory event based on the comparison between the respiratory data and the comparative respiratory data.

2. The method of claim 1, wherein the detection window is a moving detection window and the comparative detection window is a moving comparative detection window.

3. The method of claim 2, wherein comparing the respiratory data to the comparative respiratory data comprises:
determining a measure of variation of the respiratory data during the moving detection window;
determining a comparative measure of variation of the comparative respiratory data during the moving comparative detection window; and
comparing the measure of variation and the comparative measure of variation,
wherein the obstructive respiratory event is detected based on the comparison between the measure of variation and the comparative measure of variation.

4. The method of claim 3, wherein the measure of variation is one of a standard deviation or a variance and the comparative measure of variation is one of a comparative standard deviation or a comparative variance.

5. The method of claim 1, further comprising:
detecting a body position change of the patient; and
in response to detecting the body position change, recapturing the respiratory data and the comparative respiratory data prior to comparing the respiratory data to the comparative respiratory data.

6. The method of claim 1, wherein the respiratory data is continuously compared to the comparative respiratory data and detecting the obstructive respiratory event comprises:
detecting an onset of a respiratory event at a first time based on the comparison between the respiratory data and the comparative respiratory data;
detecting an end of the respiratory event at a second time based on the comparison between the respiratory data and the comparative respiratory data; and
determining that the respiratory event is the obstructive respiratory event based on a time period between the first time and the second time being above a threshold time period.

7. The method of claim 6, wherein detecting the onset of the respiratory event includes detecting a ratio between the respiratory data and the comparative respiratory data falling below a first threshold, and detecting the end of the respiratory event includes detecting the ratio rising above a second threshold, wherein the second threshold is higher than the first threshold.

8. The method of claim 1, wherein detecting the obstructive respiratory event comprises:
detecting a baseline number of obstructive respiratory events during a baseline period, in which no stimulation or a first stimulation intensity is delivered to the patient; and
detecting one or more parameter-specific numbers of obstructive respiratory events during one or more stimulation periods, in which one or more second stimulation intensities are delivered to the patient, the one or more second stimulation intensities being different than the first stimulation intensity.

9. The method of claim 8, further comprising displaying a reduction in obstructive respiratory events during the one or more stimulation periods compared to the baseline period.

10. The method of claim 8, further comprising:
comparing the baseline number of obstructive respiratory events to the one or more parameter-specific numbers of obstructive respiratory events;
identifying one or more stimulation parameters corresponding to a reduction in obstructive respiratory events; and
delivering stimulation to the patient via the implantable medical device using the one or more stimulation parameters.

11. The method of claim 8, further comprising tracking, for each detected obstructive respiratory event during the baseline period and the one or more stimulation periods, one or more of a body position of the patient or a sleep stage of the patient.

12. The method of claim 1, wherein the respiratory data and the comparative respiratory data comprise acceleration data that is associated with chest acceleration of the patient and indicative of respiration of the patient.

13. An implantable medical device comprising:
a sensor;
a stimulation lead having a plurality of electrodes; and
a pulse generator configured to deliver stimulation to one or more electrodes of the plurality of electrodes of the stimulation lead, the pulse generator comprising a processing circuit including a processor and a memory, the memory having instructions stored thereon that, when executed by the processor, cause the processor to:
capture respiratory data and comparative respiratory data of a patient using the sensor, the respiratory data captured during a detection window and the comparative respiratory data captured during a comparative detection window at least partially preceding the detection window;
compare the respiratory data to the comparative respiratory data;
detect obstructive respiratory events during each of a baseline period and one or more stimulation periods based on the comparison of the respiratory data to the comparative respiratory data;
identify one or more stimulation parameters corresponding to a reduction in the obstructive respiratory events; and
deliver stimulation to the patient using the one or more stimulation parameters.

14. The implantable medical device of claim 13, wherein the one or more stimulation parameters include at least one of a stimulation current amplitude, a stimulation frequency, a stimulation duty cycle, a stimulation pulse width, or an activated electrode selection.

15. The implantable medical device of claim 13, wherein the detection window is a moving detection window, the comparative detection window is a moving comparative detection window, and comparing the respiratory data to the comparative respiratory data comprises:
determining a measure of variation of the respiratory data during the moving detection window;
determining a comparative measure of variation of the comparative respiratory data during the moving comparative detection window; and
comparing the measure of variation and the comparative measure of variation,
wherein the obstructive respiratory events are detected based on the comparison between the measure of variation and the comparative measure of variation.

16. The implantable medical device of claim 13, wherein the instructions, when executed by the processor, further cause the processor to:
detect a body position change of the patient; and
in response to detecting the body position change, recapture the respiratory data and the comparative respiratory data prior to comparing the respiratory data to the comparative respiratory data.

17. The implantable medical device of claim 13, wherein detecting the obstructive respiratory events comprises:
detecting an onset of a respiratory event at a first time based on the comparison between the respiratory data and the comparative respiratory data;
detecting an end of the respiratory event at a second time based on the comparison between the respiratory data and the comparative respiratory data; and
determining that the respiratory event is an obstructive respiratory event based on a time period between the first time and the second time being above a threshold time period.

18. One or more non-transitory computer-readable media comprising instructions executable by one or more processors to:
capture respiratory data and comparative respiratory data of a patient using a sensor of an implantable medical device, the respiratory data captured during a detection window and the comparative respiratory data captured during a comparative detection window at least partially preceding the detection window;
compare the respiratory data to the comparative respiratory data;
detect obstructive respiratory events during each of a baseline period, in which no stimulation or a first stimulation intensity is delivered to the patient, and one or more stimulation periods, in which one or more second stimulation intensities are delivered to the patient, based on the comparison of the respiratory data to the comparative respiratory data; and
identify one or more stimulation parameters corresponding to a reduction in the obstructive respiratory events based on the detected obstructive respiratory events during each of the baseline period and the one or more stimulation periods.

19. The one or more non-transitory computer-readable media of claim 18, wherein the respiratory data and the comparative respiratory data are continuously captured, the detection window is a moving detection window, and the comparative detection window is a moving comparative detection window.

20. The one or more non-transitory computer-readable media of claim 19, wherein comparing the respiratory data to the comparative respiratory data comprises:
determining a measure of variation of the respiratory data during the moving detection window;
determining a comparative measure of variation of the comparative respiratory data during the moving comparative detection window; and
comparing the measure of variation and the comparative measure of variation,
wherein the obstructive respiratory events are detected based on the comparison between the measure of variation and the comparative measure of variation.
